(19) United States
(12) Patent Application Publication
(10) Pub. No.: US 2018/0372746 A1
(43) Pub. Date: Dec. 27, 2018

(54) HIGH-THROUGHPUT SCREENING ASSAY

(71) Applicant: BellBrook Labs, Madison, WI (US)

(72) Inventors: Robert G. Lowery, Madison, WI (US); Meera Kumar, Madison, WI (US)

(21) Appl. No.: 16/014,240

(22) Filed: Jun. 21, 2018

Related U.S. Application Data
(60) Provisional application No. 62/523,469, filed on Jun. 22, 2017.

(51) Int. Cl. G01N 33/573; G01N 21/64; G01N 21/76

(52) U.S. Cl. CPC G01N 33/573; G01N 21/6428; G01N 2021/6439; G01N 2333/9125; G01N 2400/00; G01N 21/763

(57) ABSTRACT

Methods and materials for development of high-throughput screening assays for detection of cyclic GMP (cGAMP) and/or cyclic GMP-AMP synthase (cGAS) activity are provided by this invention.

### CROSS-REFERENCE TO RELATED APPLICATIONS

Benefit of priority is claimed to U.S. Provisional Application Ser. No. 62/523,469, filed Jun. 22, 2017, the disclosure of which is incorporated herein by reference in its entirety.

### BACKGROUND OF THE INVENTION

### Field of invention

The present invention relates to methods and materials for high-throughput screening assays for measuring cyclic GMP-AMP synthase (cGAS) activity and/or detecting G(2′-5′)pA(3′-5′)p (cGAMP).

### Description of Related Art

Cyclic GMP-AMP synthase (cGAS) (UniProtKB-Q8N884) is a recently discovered enzyme that acts as a foreign DNA sensor to elicit an immune response to pathogens via activation of the stimulator of interferon genes (STING) receptor. Shortly after its discovery in 2013, aberrant activation of cGAS by self-DNA was shown to underlie debilitating and sometimes fatal autoimmune diseases, such as systemic lupus erythematosus (SLE), scleroderma, and Aicardi-Goutieres Syndrome (AGS). Knockout studies in animal models have clearly indicated that inhibiting cGAS is a promising approach for therapeutic intervention. Very recent studies have also implicated the cGAS-STING pathway in the innate immune response to tumors; thus, enhancement of cGAS activity is an emerging strategy for cancer immunotherapy.

However, there are no HTS-compatible assay methods for detection of cGAS enzyme activity, and development of a homogenous assay presents a considerable challenge, as it requires selective detection of the cyclic dinucleotide product, cGAMP (FIG. 1), in the presence of the substrates ATP and GTP. Researchers currently are relying on radioassays coupled with thin-layer-chromatography for biochemical cGAS assays and LC-MS for detection of cGAMP in cell lysates or tissue samples. Therefore, there remains a need to develop sensors conducive to performing HTS assays for measuring cGAS activity and detecting cGAMP.

### SUMMARY OF THE INVENTION

It is against the above background that the present invention provides certain advantages and advancements over the prior art.

Although this invention as described herein is not limited to specific advantages or functionalities (such as for example, the ability to measure G(2′-5′)pA(3′-5′)p (cGAMP) produced in an enzymatically catalyzed reaction and/or cyclic GMP-AMP synthase (cGAS) activity in a competitive assay method, a homogenous assay method, an assay method having low nanomolar sensitivity, an assay method allowing for direct detection of cGAMP, an assay method operable in endpoint or continuous mode; and/or a high-throughput screening (HTS) assay method), the invention provides a method for measuring G(2′-5′)pA(3′-5′)p (cGAMP) produced in an enzymatically catalyzed reaction, comprising:
(a) contacting, in a first reaction, a biological sample with an enzyme and a first substrate molecule to form a first product;
(b) contacting, in a second reaction, the first product with a second substrate molecule in the presence of a first catalytically active enzyme to form a second product and a third product; and
(c) detecting the second and the third products by an antibody;
thereby measuring the produced cGAMP.

In one aspect of the methods for measuring G(2′-5′)pA(3′-5′)p (cGAMP) produced in an enzymatically catalyzed reaction disclosed herein:
(a) the first substrate molecule is adenosine triphosphate (ATP) and/or guanosine triphosphate (GTP);
(b) the second substrate molecule comprises a phosphate molecule;
(c) the first catalytically active enzyme is a phosphodiesterase, comprising snake venom phosphodiesterase-1 (SVPDE) and/or ectonucleotide pyrophosphatase/phosphodiesterase 1 (ENPP1);
(d) the enzyme catalyzing step (a) comprises cyclic GMP-AMP synthase (cGAS) activity;
(e) the first product is cyclic guanosine monophosphate-adenosine monophosphate (cGAMP);
(f) the second product is adenosine monophosphate (AMP);
(g) the third product is guanosine monophosphate (GMP); and
(h) the antibody is an AMP/GMP-specific antibody.

In one aspect, the methods for measuring G(2′-5′)pA(3′-5′)p (cGAMP) produced in an enzymatically catalyzed reaction disclosed herein further comprise:
(d) contacting, in a third reaction, the second product and the third product in the presence of a second catalytically active enzyme to form a fourth product and a fifth product.

In one aspect of the methods for measuring G(2′-5′)pA(3′-5′)p (cGAMP) produced in an enzymatically catalyzed reaction disclosed herein:
(a) the second catalytically active enzyme comprises AMP/GMP kinase;
(b) the fourth product is adenosine diphosphate (ADP); and
(c) the fifth product is guanosine diphosphate (GDP).

In one aspect of the methods for measuring G(2′-5′)pA(3′-5′)p (cGAMP) produced in an enzymatically catalyzed reaction disclosed herein, the formation of the fourth product is detected by:
(a) contacting, in a first reaction, the fourth product and a third substrate molecule in the presence of a third catalytically active enzyme to form a sixth product;
(b) contacting, in a second reaction, the sixth product with a bioluminescent agent and/or a fluorescent agent in the presence of a fourth catalytically active enzyme;
   thereby generating a signal, comprising a bioluminescent signal and/or a fluorescent signal; and
(c) measuring the signal.

In one aspect of the methods for measuring G(2′-5′)pA(3′-5′)p (cGAMP) produced in an enzymatically catalyzed reaction disclosed herein:
(a) the third substrate molecule is phosphoenolpyruvate;
(b) the third catalytically active enzyme is pyruvate kinase and/or pyruvate oxidase;
(c) the sixth product is adenosine triphosphate (ATP) and/or hydrogen peroxide;
(d) the fourth catalytically active enzyme is luciferase;
(e) the bioluminescent agent is luciferin; and
(f) the fluorescent agent comprises Amplex Red, fluorescein, mCherry fluorescent protein, cyanine dyes, TRITC, pacific blue, and/or pacific orange.

In one aspect, the methods for measuring G(2′-5′)pA(3′-5′)p (cGAMP) produced in an enzymatically catalyzed reaction disclosed herein further comprise detecting cGAMP in the biological sample; thereby measuring cGAMP produced in the reaction.

In one aspect of the methods for measuring G(2′-5′)pA(3′-5′)p (cGAMP) produced in an enzymatically catalyzed reaction disclosed herein, the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

The invention also provides a method for measuring cyclic GMP-AMP synthase (cGAS) activity, comprising:
(a) contacting, in a first reaction, a biological sample with an enzyme and a first substrate molecule to form a first product; and
(b) contacting, in a second reaction, the first product with a second substrate molecule in the presence of a first catalytically active enzyme to form a second product and a third product; and
(c) detecting the second and the third products by an antibody;
thereby measuring the cGAS activity.

In one aspect of the methods for measuring cyclic GMP-AMP synthase (cGAS) activity disclosed herein:
(a) the first substrate molecule is adenosine triphosphate (ATP) and/or guanosine triphosphate (GTP);
(b) the second substrate molecule comprises a phosphate molecule;
(c) the first catalytically active enzyme is a phosphodiesterase, comprising snake venom phosphodiesterase-1 (SVPDE) and/or ectonucleotide pyrophosphatase/phosphodiesterase 1 (ENPP1);
(d) the enzyme catalyzing step (a) comprises cyclic GMP-AMP synthase (cGAS) activity;
(e) the first product is cyclic guanosine monophosphate-adenosine monophosphate (cGAMP);
(f) the second product is adenosine monophosphate (AMP);
(g) the third product is guanosine monophosphate (GMP); and
(h) the antibody is an AMP/GMP-specific antibody.

In one aspect, the methods for measuring cyclic GMP-AMP synthase (cGAS) activity disclosed herein further comprise:
(d) contacting, in a third reaction, the second product and the third product in the presence of a second catalytically active enzyme to form a fourth product and a fifth product.

In one aspect of the methods for measuring cyclic GMP-AMP synthase (cGAS) activity disclosed herein:
(a) the second catalytically active enzyme comprises AMP/GMP kinase;
(b) the fourth product is adenosine diphosphate (ADP); and
(c) the fifth product is guanosine diphosphate (GDP).

In one aspect of the methods for measuring cyclic GMP-AMP synthase (cGAS) activity disclosed herein, the formation of the fourth product is detected by:
(a) contacting, in a first reaction, the fourth product and a third substrate molecule in the presence of a third catalytically active enzyme to form a sixth product;
(b) contacting, in a second reaction, the sixth product with a bioluminescent agent and/or a fluorescent agent in the presence of a fourth catalytically active enzyme;
   thereby generating a signal, comprising a bioluminescent signal and/or a fluorescent signal; and
(c) measuring the signal.

In one aspect of the methods for measuring cyclic GMP-AMP synthase (cGAS) activity disclosed herein:
(a) the third substrate molecule is phosphoenolpyruvate;
(b) the third catalytically active enzyme is pyruvate kinase and/or pyruvate oxidase;
(c) the sixth product is adenosine triphosphate (ATP) and/or hydrogen peroxide;
(d) the fourth catalytically active enzyme is luciferase;
(e) the bioluminescent agent is luciferin; and
(f) the fluorescent agent comprises Amplex Red, fluorescein, mCherry fluorescent protein, cyanine dyes, tetramethylrhodamine (TRITC), pacific blue, and/or pacific orange.

In one aspect of the methods for measuring cyclic GMP-AMP synthase (cGAS) activity disclosed herein further comprise detecting cGAMP in the biological sample; thereby measuring cyclic GMP-AMP synthase (cGAS) activity.

In one aspect of the methods for measuring cyclic GMP-AMP synthase (cGAS) activity disclosed herein, the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

The invention also provides a method for measuring G(2′-5′)pA(3′-5′)p (cGAMP) produced in an enzymatically catalyzed reaction and/or cyclic GMP-AMP synthase (cGAS) activity, comprising:
(a) contacting a biological sample with a substrate molecule and an agent in the presence of a catalytically active enzyme;
(b) measuring a signal; and
(c) detecting cGAMP in the biological sample; thereby measuring cGAMP produced in the reaction and/or cyclic GMP-AMP synthase (cGAS) activity.

In one aspect of the methods for measuring G(2′-5′)pA(3′-5′)p (cGAMP) produced in an enzymatically catalyzed reaction and/or cyclic GMP-AMP synthase (cGAS) activity disclosed herein:
(a) the substrate molecule comprises adenosine triphosphate (ATP), guanosine triphosphate (GTP), a phosphate molecule, and/or phosphoenolpyruvate;
(b) the catalytically active enzyme comprises a phosphodiesterase, AMP/GMP kinase, pyruvate kinase, pyruvate oxidase, luciferase, and/or peroxidase;
(c) the agent comprises a bioluminescent agent, comprising luciferin or a fluorescent agent, comprising Amplex Red; and
(d) the signal is a bioluminescent signal or a fluorescent signal.

In one aspect of the methods for measuring G(2′-5′)pA(3′-5′)p (cGAMP) produced in an enzymatically catalyzed reaction and/or cyclic GMP-AMP synthase (cGAS) activity disclosed herein, the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

The invention also provides a kit for:
(a) detecting and measuring cGAMP produced in an enzymatically catalyzed reaction, comprising adenosine triphosphate (ATP), guanosine triphosphate (GTP), a phosphate molecule, phosphoenolpyruvate, a phosphodiesterase, AMP/GMP kinase, pyruvate kinase, luciferase, and a bioluminescent agent;
(b) detecting and measuring cGAMP produced in an enzymatically catalyzed reaction, comprising adenosine triphosphate (ATP), guanosine triphosphate (GTP), a phosphate molecule, phosphoenolpyruvate, a phosphodiesterase, AMP/GMP kinase, pyruvate kinase, pyruvate oxidase, peroxidase, and a fluorescent agent;
(c) measuring cyclic GMP-AMP synthase (cGAS) activity, comprising adenosine triphosphate (ATP), guanosine triphosphate (GTP), a phosphate molecule, phosphoenolpyruvate, a phosphodiesterase, AMP/GMP kinase, pyruvate kinase, luciferase, and a bioluminescent agent;
(d) measuring cyclic GMP-AMP synthase (cGAS) activity, comprising adenosine triphosphate (ATP), guanosine triphosphate (GTP), a phosphate molecule, phosphoenolpyruvate, a phosphodiesterase, AMP/GMP kinase, pyruvate kinase, pyruvate oxidase, peroxidase, and a fluorescent agent;
(e) detecting and measuring cGAMP produced in an enzymatically catalyzed reaction, comprising an antibody against AMP/GMP, wherein the antibody has the ability to preferentially recognize AMP/GMP in the presence of substrate molecules, and wherein the antibody specifically binds AMP/GMP; and/or
(f) measuring cyclic GMP-AMP synthase (cGAS) activity, comprising an antibody against AMP/GMP, wherein the antibody has the ability to preferentially recognize AMP/GMP in the presence of substrate molecules, and wherein the antibody specifically binds AMP/GMP.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic structure of cGAMP.

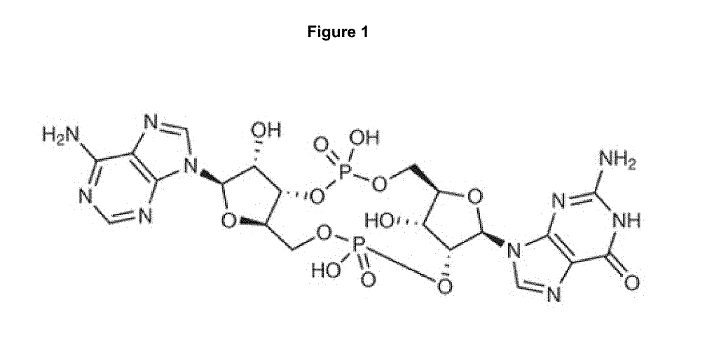

FIG. 2 is a schematic for a cGAMP enzymatic assay with detection of cGAMP by antibody-based methods.

FIG. 3 is a schematic for a cGAMP enzymatic assay with detection of cGAMP by bioluminescence-based methods.

FIG. 4 is a schematic for a cGAMP enzymatic assay with detection of cGAMP by fluorescence-based methods.

Skilled artisans will appreciate that elements in the figures are illustrated for simplicity and clarity and have not necessarily been drawn to scale. For example, the dimensions of some of the elements in the figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide,” “nucleotide,” “oligonucleotide,” and “nucleic acid” can be used interchangeably to refer to nucleic acid comprising DNA, RNA, derivatives thereof, or combinations thereof.

As used herein, the terms “homogenous assay,” “homogenous format,” and “homogenous detection” can be used to refer to detection of an analyte by a simple mix and read procedure. A homogenous assay does not require steps such as sample washing or sample separation steps. Examples of homogeneous assays include TR-FRET, FP, FI, bioluminescence, and luminescence-based assays.

As used herein, the term “bioluminescence agent” includes, but is not limited to luciferin.

As used herein, the term “fluorescence agent” includes, but is not limited to, Amplex Red, fluorescein, mCherry fluorescent protein, cyanine dyes, tetramethylrhodamine (TRITC), pacific blue, and pacific orange.

As used herein, the term “and/or” is utilized to describe multiple components in combination or exclusive of one another. For example, “x, y, and/or z” can refer to “x” alone, “y” alone, “z” alone, “x, y, and z,” “(x and y) or z,” “x and (y or z),” or “x or y or z.”

As used herein, the term “contacting” refers to the act of touching, making contact, or of bringing to immediate or close proximity, including at the cellular or molecular level, for example, to bring about a physiological reaction, a chemical reaction, or a physical change, e.g., in a solution, in a test mixture, in vitro, or in vivo.

The term “catalytically active enzyme” as used herein refers to at least one of a kinase, a synthase, a pyrophosphatase/phosphodiesterase, a pyrophosphatase, a phosphodiesterase, a peroxidase, an oxidase, a sulfotransferase, among others. Examples of a catalytically active enzyme can include, but is not limited to, ectonucleotide pyrophosphatase/phosphodiesterase-1 (ENPP1), snake venom phosphodiesterase-1 (SVPDE), AMP/kinase, pyruvate kinase, and pyruvate oxidase, among others.

The term “catalytic activity” as used herein refers to a chemical catalytic activity, an enzymatic activity, or a combination thereof.

The term “cofactor” as used herein refers to a non-protein chemical compound or metallic ion that is required for a protein's biological activity to happen. For example a cofactor can include, but is not limited to, a vitamin, thiamine pyrophosphate (TPP), lipoamide, flavin adenine dinucleotide (FAD), nicotinamide adenine dinucleotide (NAD+), heme, coenzyme A (CoA), magnesium, copper, manganese, and iron-sulfur clusters, among others.

The term “substrate molecule” as used herein refers to a molecule upon which an enzyme acts. Enzymes catalyze chemical reactions involving the substrate(s) and the substrate molecule is transformed into one or more products. Examples of suitable substrate molecules can include not only nucleotides, but also phosphoenolpyruvic acid (PEP), s-adenosyl methionine and acetyl-CoA, among others.

The term “high throughput screening” or “HTS” as used herein refers to the testing of many thousands of molecules (or test compounds) for their effects on the function of a protein. In the case of group transfer reaction enzymes many molecules may be tested for effects on their catalytic activity. HTS methods are known in the art and they are generally performed in multiwell plates with automated liquid handling and detection equipment; however it is envisioned that the methods of the invention may be practiced on a microarray or in a microfluidic system.

cGAS-cGAMP-STING Pathway

The cGAS-cGAMP-STING pathway activates the immune system in response to foreign DNA. The presence of DNA in the cytosol of eukaryotic cells is an indicator of infection or cellular damage, and it elicits a strong immune response, including type I interferon (IFN) induction. The STING protein was shown to mediate this response via the NF-KB and IRF3 transcription pathways in 2008, and bacterial cyclic dinucleotides were identified as STING agonists in 2011 (Burdette D. L., et al., STING is a direct innate immune sensor of cyclic di-GMP. Nature. 2011; 478(7370):515-8; Ishikawa H. and Barber G. N., STING is an endoplasmic reticulum adaptor that facilitates innate immune signaling. Nature. 2008; 455(7213):674-8). The mechanism of STING activation by DNA remained a mystery until 2013 when cyclic GAMP synthase (cGAS) was identified by two groups as the sensor for cytosolic DNA (Sun L., et al., Cyclic GMP-AMP synthase is a cytosolic DNA sensor that activates the type I interferon pathway. Science (New York, N.Y.). 2013; 339(6121):786-91; Wu J., et al., Cyclic GMP-AMP is an endogenous second messenger in innate immune signaling by cytosolic DNA. Science (New York, N.Y.). 2013; 339(6121):826-30). Double strand DNA and DNA:RNA hybrids bind to a specific site on cGAS in a non-sequence-dependent manner and activate its catalytic activity, resulting in the production of a unique cyclic nucleotide G(2′-5′)pA(3′-5′)p (cGAMP) from ATP and GTP precursors (Ablasser A., et al., cGAS produces a 2′-5′-linked cyclic dinucleotide second messenger that activates STING. Nature. 2013; 498(7454):380-4; Diner E. J., et al., The innate immune DNA sensor cGAS produces a noncanonical cyclic dinucleotide that activates human STING. Cell Reports. 2013; 3(5):1355-61; Kato K., et al., Structural and functional analyses of DNA-sensing and immune activation by human cGAS. PloS one. 2013; 8(10):e76983; Mankan A. K., et al., Cytosolic RNA:DNA hybrids activate the cGAS-STING axis. The EMBO Journal. 2014, 33(24):2937-46). cGAMP in turn binds to the STING protein to initiate induction of the type I IFN pathway (Cai X., et al., The cGAS-cGAMP-STING pathway of cytosolic DNA sensing and signaling. Molecular Cell. 2014; 54(2):289-96). The mixed 2′-5′ and 3′-5′ phosphodiester linkages in cGAMP are not found in any known bacterial cyclic dinucleotides. Though other DNA sensors have been identified in specific types of cell types, the cGAS-cGAMP-STING pathway appears to be essential for DNA-mediated immune response irrespective of cell type or DNA sequence (Cai, et al., 2014).

Blocking cGAS activity prevents aberrant activation of inflammatory pathways in autoimmune diseases. Cytoplasmic DNA and RNA is normally rapidly degraded by nucleases, and loss of function mutations in these enzymes are linked to autoimmune diseases such as Aicardi-Goutieres Syndrome (AGS), systemic lupus erythematosus (SLE), scleroderma, Sjogren's syndrome (SS), and retinal vasculopathy (Gao D., et al., Activation of cyclic GMP-AMP synthase by self-DNA causes autoimmune diseases. Proceedings of the National Academy of Sciences of the United States of America. 2015; 112(42):E5699-705; Gray E. E., et al., Cutting Edge: cGAS Is Required for Lethal Autoimmune Disease in the Trex1-Deficient Mouse Model of Aicardi-Goutieres Syndrome. Journal of Immunology (Baltimore, Md.:1950). 2015; 195(5):1939-43; Mackenzie K. J., et al., Ribonuclease H2 mutations induce a cGAS/STING-dependent innate immune response. The EMBO Journal. 2016; 35(8):831-44). Elevated expression of IFN-stimulated genes (ISGs) is a hallmark of autoimmune diseases, and multiple lines of evidence have shown that activation of the cGAS-cGAMP-STING pathway by cytoplasmic nucleic acids DNA triggers the pathogenic IFN responses (Cai, et al., 2014). Nucleic acid-triggered autoimmune diseases cause significant pain and suffering and shorten life spans for hundreds of thousands of people in the U.S. alone. Though often not directly fatal, they are frequently disabling, and can lead to lethal complications. For example, when scleroderma progresses to organs other than skin, the 10 year survival decreases from 75% to 55%, with most deaths from kidney, heart and lung complications. SLE significantly increases the risk of cardiovascular diseases, and 20% of patients die within 15 years of diagnosis. Treatment of these diseases is currently limited to management of symptoms.

Knocking out or mutating the nucleases that clear self-DNA or RNA in mice recapitulates autoimmune disease phenotypes and IFN pathway activation. Recently these models have been used to investigate the involvement of the cGAS-cGAMP-STING pathway—and specifically cGAS—in pathogenesis. Trex1 is a cytosolic DNA exonuclease that is mutated in AGS, SLE, and other disorders, and Trex1−/− mice exhibit lethal autoimmune disease (Rice G. I., et al., Human disease phenotypes associated with mutations in TREX1. Journal of Clinical Immunology. 2015; 35(3):235-43). Genetic ablation of cGAS—or STING—protects against lethality and eliminates the important autoimmune phenotypes, including ISG induction, autoantibody production, T-cell activation (Gao, et al., 2015; Gray, et al., 2015; Mackenzie, et al., 2016; Yang Y. G., et al., Trex1 exonuclease degrades ssDNA to prevent chronic checkpoint activation and autoimmune disease. Cell. 2007; 131(5):873-86). Furthermore, elevated levels of cGAMP were detected in the Trex1 deficient mice, and knocking out cGAS prevented its accumulation (Gao, et al., 2015). Similar results were observed when cGAS was eliminated in mice lacking DNasell, a lysosomal endonuclease that clears DNA from dead cells (Gao, et al., 2015). RNA:DNA hybrids, which can be generated during aberrant DNA replication, can also induce a cGAS-cGAMP-STING dependent type I IFN response in cells, and activation of cGAS by RNA/DNA hybrids was demonstrated in in vitro biochemical assays (Mankan, et al., 2014). Mutations that impair the function of RNase H2, the major enzyme responsible for clearing DNA:RNA hybrids, are the predominant cause of AGS and are found less frequently in SLE (Mackenzie, et al,. 2016). Very recently, two groups showed that mice lacking functional RNaseH2 show strong ISG transcript upregulation, and elimination of cGAS—or STING—in the RNaseH2 deficient mice rescued the inflammatory phenotypes (Mackenzie, et al,. 2016; Yang, et al., 2007; Pokatayev V., et al., RNase H2 catalytic core Aicardi-Goutieres syndrome-related mutant invokes cGAS-STING innate immune-sensing pathway in mice. The Journal of Experimental Medicine. 2016; 213 (3):329-36).

The cGAS-cGAMP-STING pathway has also been implicated as playing an important role in idiopathic autoimmune diseases. Plasmacytoid dendritic cells (pDCs) protect against viral and bacterial infections by recognizing single stranded RNA and unmethylated DNA sequences via their toll-like receptors and producing type I IFNs. Continuous stimulation of pDCs by nucleic acid immune complexes is believed to be a major driver of autoimmune disease progression, most demonstrably in SLE (Ronnblom L., et al., Role of natural interferon-alpha producing cells (plasmacytoid dendritic cells) in autoimmunity. Autoimmunity. 2003; 36(8):463-72). The cGAS-cGAMP-STING pathway was shown to be required for DNA-induced type 1 IFN response in pDCs: activation of STING by cGAMP elicited the IFN response in pDCs and STING knockdown abolished the response (Bode C., et al., Human plasmacytoid dentritic cells elicit a Type I Interferon response by sensing DNA via the cGAS-STING signaling pathway).

The cGAS-cGAMP-STING pathway has also been implicated in idiopathic infantile arterial calcification. cGAMP is hydrolyzed by a phosphodiesterase, ecto-nucleotide pyrophosphatase/phosphodiesterase (ENPP1), into adenosine monophosphate (AMP) and guanosine monophosphate (GMP) (Tao, et al., cGAS-cGAMP-STING: The three musketeers of cytosolic DNA sensing and signaling. IUBMB Life. 2016; 68(11):858-870). However, inactivating mutations in ENPP1 gene leads to a decrease in inorganic pyrophosphate (PPi), which is a potent inhibitor of calcium deposition in the vessel wall. Consequently, mutations in the ENPP1 gene lead to calcification of the internal elastic lamina of muscular arteries and stenosis due to myintimal proliferation (Rutsch, et al., PC-1 nucleoside triphosphate pyrophosphohydrolase deficiency in idiopathic infantile arterial calcification. Am J Pathol. 2001; 158(2):543-54; Rutsch, et al., Mutations in ENPP1 are associated with ‘idiopathic’ infantile arterial calcification. Nat Genet. 2003; 34, 379-381). These results further build the case for targeting cGAS for autoimmune diseases that affect quality and longevity of life for hundreds of thousands of people in the U.S. alone. Taken together, these animal and cellular studies make a compelling case for blocking cGAMP production with a small molecule inhibitor as a therapeutic intervention for SLE, AGS, and other autoimmune diseases.

Modulation of the cGAS-cGAMP-STING pathway is also a promising approach for cancer immunotherapy (Lemos H., et al., STING, nanoparticles, autoimmune disease and cancer: a novel paradigm for immunotherapy? Expert Review of Clinical Immunology. 2015; 11(1):155-65). Immunotherapy approaches such as immune checkpoint blockade are transforming oncology, however many patients do not respond to existing agents, and alternatives are needed. In vitro and in vivo studies have shown that activation of the cGAS-STING nexus by DNA from tumor cells and/or dead host cells is a major mechanism for activation of the innate immune response in the tumor microenvironment (Ohkuri T., et al., STING contributes to antiglioma immunity via triggering type I IFN signals in the tumor microenvironment. Cancer Immunology Research. 2014; 2(12):1199-208; Woo S R, et al., STING-dependent cytosolic DNA sensing mediates innate immune recognition of immunogenic tumors. Immunity. 2014, 41(5):830-42). Moreover, STING agonists have shown anti-tumor activity in mouse models of melanoma, glioma, colon, breast, and prostate cancers (Ohkuri, et al., 2014; Corrales L., et al., Direct Activation of STING in the Tumor Microenvironment Leads to Potent and Systemic Tumor Regression and Immunity. Cell Reports. 2015; 11(7):1018-30). Similarly, small molecules that activate cGAS—possibly by binding to the DNA binding site—could potentially be used to stimulate the recruitment of immune cells in the tumor microenvironment.

G(2′-5′)pA(3′-5′)p (cGAMP) Assay

Development of small molecule cGAS modulators is hampered by the lack of HTS assays. The only method for detecting cGAS activity that has been reported thus far is semi-quantitative determination of radioactive cGAMP formation from radiolabeled ATP and GTP precursors (Ablasser, et al., 2013; Diner, et al., 2013; Kranzusch P. J., et al., Structure of human cGAS reveals a conserved family of second-messenger enzymes in innate immunity. Cell Reports. 2013; 3(5):1362-8). Aside from the undesirability of radioassays, this method requires separation by thin layer chromatography, and thus is not amenable to HTS. Development of a competitive displacement assay for cGAMP using recombinant STING and fluorescent tracers has been hampered because attachment of fluors to cGAMP disrupted its binding to STING.

The ability to readily detect cGAMP without chromatographic separation and/or radioactive labeling would greatly accelerate basic research and drug discovery targeting the pathway. The homogenous, fluorescent and/or bioluminescent assays described herein for detecting cGAMP can have a profound impact on basic research, drug discovery, and translational studies targeting STING/cGAS for autoimmune diseases and cancer immunotherapy.

Development of cGAMP assays with fluorescent and/or bioluminescent readouts enables mix-and-read cGAS enzyme assays with the sensitivity and robustness required for automated HTS platforms. Applicants have previously developed the use of homogenous immunodetection of nucleotides for HTS enzyme assays (Lowery R. G., et al., Transcreener: screening enzymes involved in covalent regulation. Expert Opin Ther Targets. 2006; 10(1):179-90). Applicants' Transcreener assays for ADP, GDP, UDP and AMP/GMP have been broadly used in tens of millions of wells of pharma, biotech, and academic HTS for diverse enzyme targets including kinases, ATPases, GTPases, and glyscosyltransferases (Huss K. L., et al., Development of a Transcreener kinase assay for protein kinase A and demonstration of concordance of data with a filter-binding assay format. J Biomol Screen. 2007; 12(4):578-84; Reichman M., et al., A High-Throughput Assay for Rho Guanine Nucleotide Exchange Factors Based on the Transcreener GDP Assay. J Biomol Screen. 2015; 20(10):1294-9). See, also, U.S. Pat. No. 7,332,278, U.S. Pat. No. 7,355,010, U.S. Pat. No. 7,847,066, U.S. Pat. No. 7,378,505, and U.S. Pat. No. 8,088,897, which have been incorporate herein by reference in their entirety.

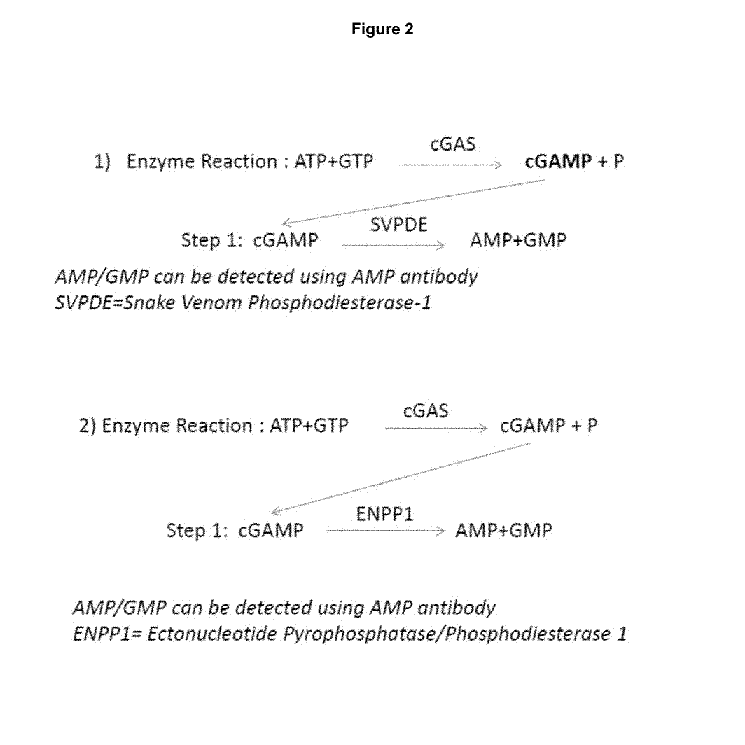

To enable HTS efforts targeting cGAMP, advantageous approaches are developed using luminescence-based and fluorescence-based methods (see Example 2 and 3 and FIGS. 2-4). The two methods enable the detection of ADP (adenosine diphosphate) by luciferase assay or an enzyme-coupled reaction converting ADP to a fluorescent signal. The assays are homogeneous and are ideal for high throughput screening applications. Additionally, the assays have an excellent ATP tolerance and robust signal to background ratios, and compatible with unmodified peptides and whole protein substrates.

In one embodiment, a method for measuring G(2′-5′)pA(3′-5′)p (cGAMP) produced in an enzymatically catalyzed reaction is developed as follows. In a first reaction, a biological sample is contacted with substrate molecules, which are adenosine triphosphate (ATP) and guanosine triphosphate (GTP) to form a first product, cyclic guanosine monophosphate-adenosine monophosphate (cGAMP). In a second reaction, the first product is contacted with a second substrate molecule, which is a phosphate molecule, in the presence of a first catalytically active enzyme, which is a phosphodiesterase, to form a second product, which is adenosine monophosphate (AMP) and a third product, guanosine monophosphate (GMP) (see FIG. 2). Examples of the phosphodiesterase can include, but are not limited to, snake venom phosphodiesterase-1 (SVPDE) or ectonucleotide pyrophosphatase/phosphodiesterase 1 (ENPP1). In some embodiments, the second and third products are specifically detected by an AMP/GMP-specific antibody (see Example 1).

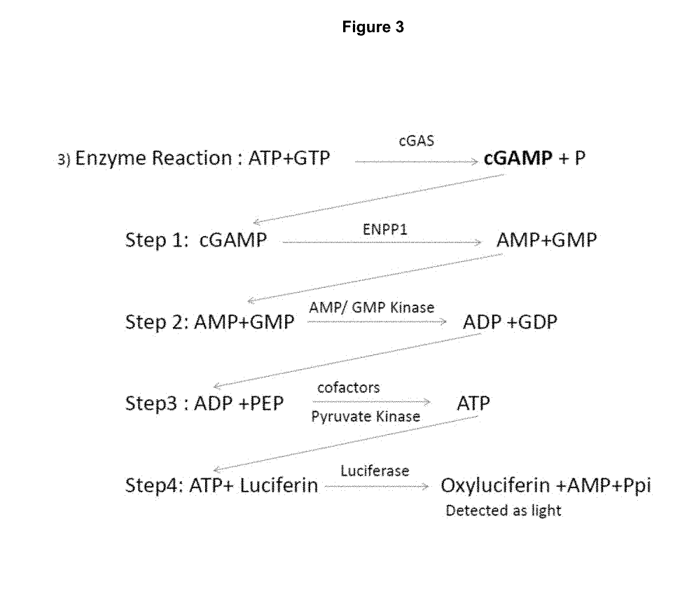

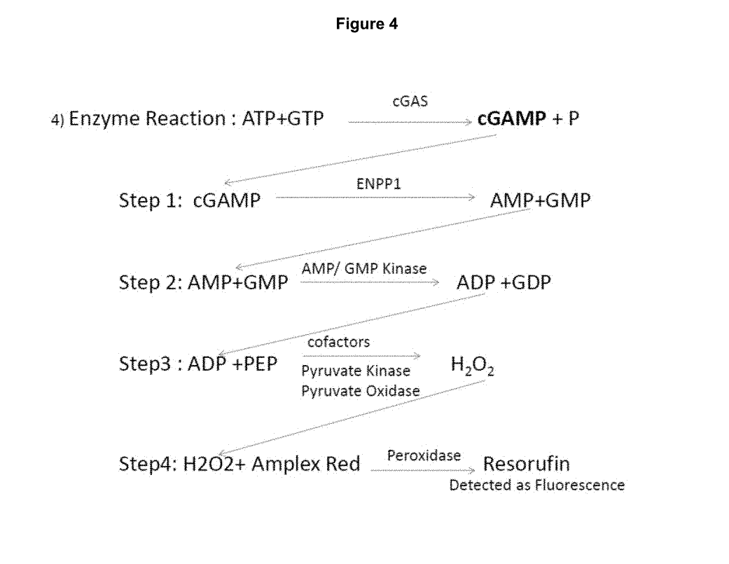

In another embodiment, a method for measuring G(2′-5′)pA(3′-5′)p (cGAMP) produced in an enzymatically catalyzed reaction is developed as follows. In a first reaction, a biological sample is contacted with substrate molecules, which are adenosine triphosphate (ATP) and guanosine triphosphate (GTP) to form a first product, cyclic guanosine monophosphate-adenosine monophosphate (cGAMP). The reaction is catalyzed by cyclic GMP-AMP synthase (cGAS). In a second reaction, the first product is contacted with a second substrate molecule, which is a phosphate molecule, in the presence of a first catalytically active enzyme, which is a phosphodiesterase, to form a second product, which is adenosine monophosphate (AMP) and a third product, guanosine monophosphate (GMP). In a third reaction, the second product and the third product are added in the presence of a second catalytically active enzyme, which is an AMP/GMP kinase, to form a fourth product, which is adenosine diphosphate (ADP), and a fifth product, guanosine diphosphate (GDP). Furthermore, the formation of the fourth product can be detected by several different reactions, two of which are shown in FIGS. 3 and 4 and described below.

In one aspect, in a first detection reaction, the fourth product and a third substrate molecule, which is phosphoenolpyruvate (PEP), are added in the presence of a third catalytically active enzyme, which is pyruvate kinase to form a sixth product, which is adenosine triphosphate (ATP). The sixth product is contacted with a bioluminescent agent, and a fourth a catalytically active enzyme (e.g., luciferase), which generates a bioluminescent signal that can be measured (see FIG. 3). Subsequently, the cGAMP produced in the reaction can be measured by detecting cGAMP in the biological sample.

In another aspect, in a second detection reaction, the fourth product and a third substrate molecule, which is phosphoenolpyruvate (PEP), are added in the presence of a third catalytically active enzyme, pyruvate kinase and/or pyruvate oxidase to form a sixth product, which is hydrogen peroxidase (H₂O₂). The sixth product is contacted with a fluorescent agent (e.g., Amplex Red), and a fourth catalytically active enzyme, peroxidase which generates a fluorescent signal that can be measured (see FIG. 4). Subsequently, the cGAMP produced in the reaction can be measured by detecting cGAMP in the biological sample.

In another embodiment, a method for measuring G(2′-5′)pA(3′-5′)p (cGAMP) produced in an enzymatically catalyzed reaction is developed by contacting a biological sample with a substrate molecule including ATP, GTP and PEP and a bioluminescent agent (e.g., luciferin), in the presence of a catalytically active enzyme which generates a signal that can be measured. The reaction is catalyzed by cGAS, a phosphodiesterase, AMP/GMP kinase, pyruvate kinase, and luciferase. Subsequently, cGAMP produced in the reaction can be measured by detecting cGAMP in the biological sample.

In another embodiment, a method for measuring G(2′-5′)pA(3′-5′)p (cGAMP) produced in an enzymatically catalyzed reaction is developed by contacting a biological sample with a substrate molecule including ATP, GTP, and PEP and a fluorescent agent (e.g., Amplex Red), in the presence of a catalytically active enzyme which generates a signal that can be measured. The reaction is catalyzed by cGAS, a phosphodiesterase, AMP/GMP kinase, pyruvate kinase, pyruvate oxidase, and peroxidase. Subsequently, cGAMP produced in the reaction can be measured by detecting cGAMP in the biological sample.

In another embodiment, a method for measuring cyclic GMP-AMP synthase (cGAS) activity is developed as follows. In a first reaction, a biological sample is contacted with substrate molecules, which are adenosine triphosphate (ATP) and guanosine triphosphate (GTP) to form a first product, cyclic guanosine monophosphate-adenosine monophosphate (cGAMP). In a second reaction, the first product is contacted with a second substrate molecule, which is a phosphate molecule, in the presence of a first catalytically active enzyme, which is a phosphodiesterase, to form a second product, which is adenosine monophosphate (AMP) and a third product, guanosine monophosphate (GMP) (see FIG. 2). The phosphodiesterase can be, for example, snake venom phosphodiesterase-1 (SVPDE) or ectonucleotide pyrophosphatase/phosphodiesterase 1 (ENPP1). In some embodiments, the second and third products are specifically detected by an AMP/GMP-specific antibody (see Example 1).

In some embodiments, a method for measuring cyclic GMP-AMP synthase (cGAS) activity is developed as follows. In a first reaction, a biological sample is contacted with substrate molecules, which are adenosine triphosphate (ATP) and guanosine triphosphate (GTP) to form a first product, cyclic guanosine monophosphate-adenosine monophosphate (cGAMP). The reaction is catalyzed by cyclic GMP-AMP synthase (cGAS). In a second reaction, the first product is contacted with a second substrate molecule, which is a phosphate molecule, in the presence of a first catalytically active enzyme, which is a phosphodiesterase, to form a second product, which is adenosine monophosphate (AMP) and a third product, guanosine monophosphate (GMP). In a third reaction, the second product and the third product are added in the presence of a second catalytically active enzyme, which is AMP/GMP kinase, to form a fourth product, which is adenosine diphosphate (ADP), and a fifth product, guanosine diphosphate (GDP). Furthermore, the formation of the fourth product can be detected by several different reactions, two of which are shown in FIGS. 3 and 4 and described below.

In one embodiment, in a first detection reaction, the fourth product and a third substrate molecule, which is phosphoenolpyruvate (PEP), are added in the presence of a third catalytically active enzyme, which is pyruvate kinase to form a sixth product, which is adenosine triphosphate (ATP). The sixth product is contacted with a bioluminescent agent, and a fourth catalytically active enzyme (e.g., luciferase), which generates a bioluminescent signal that can be measured (see FIG. 3). Subsequently, the cyclic GMP-AMP synthase (cGAS) activity in the reaction can be measured by detecting cGAMP in the biological sample.

In another embodiment, in a second detection reaction, the fourth product and a third substrate molecule, which is phosphoenolpyruvate (PEP), are added in the presence of a third catalytically active enzyme, pyruvate kinase and/ or pyruvate oxidase to form a sixth product, which is hydrogen peroxidase (H₂O₂). The sixth product is contacted with a fluorescent agent (e.g., Amplex Red), and a fourth catalytically active enzyme, peroxidase which generates a fluorescent signal that can be measured (see FIG. 4). The fluorescent agent can be, but not limited to, Amplex Red, fluorescein, mCherry, cyanine dyes, TRITC, pacific blue, and pacific orange. Subsequently, the cyclic GMP-AMP synthase (cGAS) activity in the reaction can be measured by detecting cGAMP in the biological sample.

In another embodiment, a method for measuring cyclic GMP-AMP synthase (cGAS) activity is developed by contacting a biological sample with a substrate molecule including ATP, GTP and PEP and a bioluminescent agent (e.g., luciferin) in the presence of a catalytically active enzyme which generates a signal that can be measured. The reaction is catalyzed by cGAS, a phosphodiesterase, AMP/GMP kinase, pyruvate kinase, and luciferase. Subsequently, the cyclic GMP-AMP synthase (cGAS) activity in the reaction can be measured by detecting cGAMP in the biological sample.

In another embodiment, a method for measuring cyclic GMP-AMP synthase (cGAS) activity is developed by contacting a biological sample with a substrate molecule including ATP, GTP and PEP and a fluorescent agent (e.g, Amplex Red) in the presence of a catalytically active enzyme which generates a signal that can be measured. The reaction is catalyzed by cGAS, a phosphodiesterase, AMP/GMP kinase, pyruvate kinase, pyruvate oxidase, and peroxidase. The fluorescent agent, can be but not limited to, Amplex Red, fluorescein, mCherry, cyanine dyes, TRITC, pacific blue, and pacific orange. Subsequently, the cyclic GMP-AMP synthase (cGAS) activity in the reaction can be measured by detecting cGAMP in the biological sample.

In certain embodiments, the methods are assay methods and can be a competitive assay method, a homogenous assay method, an assay method having low nanomolar sensitivity, an assay method allowing for direct detection of cGAMP, and/or an assay method operable in endpoint or continuous mode. In another embodiment, the assay method is a high-throughput screening (HTS) assay method.

In one embodiment, an assay kit for detecting and measuring cGAMP produced in an enzymatically catalyzed reaction is developed. The assay kit includes adenosine triphosphate (ATP), guanosine triphosphate (GTP), a phosphate molecule, phosphoenolpyruvate, a phosphodiesterase, AMP/GMP kinase, pyruvate kinase, pyruvate oxidase, luciferase and/or peroxidase, and a bioluminescent agent and/or a fluorescent agent.

In another embodiment, a kit for measuring cyclic GMP-AMP synthase (cGAS) activity is developed. The kit includes adenosine triphosphate (ATP), guanosine triphosphate (GTP), a phosphate molecule, phosphoenolpyruvate, a phosphodiesterase, AMP/GMP kinase, pyruvate kinase, pyruvate oxidase, luciferase and/or peroxidase, and a bioluminescent agent and/or a fluorescent agent.

In one embodiment, a kit for detecting and measuring cGAMP produced in an enzymatically catalyzed reaction is developed. The kit includes an antibody against AMP/GMP. The antibody has the ability to preferentially recognize AMP/GMP in the presence of substrate molecules, and wherein the antibody specifically binds AMP/GMP.

In another embodiment, a kit for measuring cyclic GMP-AMP synthase (cGAS) activity is developed. The kit includes an antibody against AMP/GMP. The antibody has the ability to preferentially recognize AMP/GMP in the presence of substrate molecules, and wherein the antibody specifically binds AMP/GMP.

Sensitivity requirements are determined largely by the kinetic properties of the target enzyme. Most biochemical screens are performed with substrates at their Kₘ concentrations to insure detection of competitive inhibitors. So, for measuring enzyme initial velocity, an assay must be capable of robust detection of reaction products at concentrations several-fold below the substrate Kₘ. Though the kinetic parameters of the cGAS enzyme have not yet been reported, the target disclosed herein is a robust detection of 500 nM cGAMP. This sensitivity can allow for the use of ATP and GTP concentrations as low as 5 μM, which is likely to be well below their Kₘ values given their high micromolar concentrations in the cell.

In some embodiments, the disclosed method enables detection of 500 nM cGAMP in the presence of 5 μM ATP and GTP with a Z′ greater than 0.6 and with signal stability of at least 6 hours are used. In certain embodiments, demonstration of a linear response in cGAMP formation to cGAS concentration, time, and ATP and GTP (at concentrations below Kₘ) is achieved. In some embodiments, initial velocity cGAS activity 10% consumption of substrates) is detected with a Z′ value greater than 0.6 using ATP and GTP at their Kₘ concentrations. In some aspects, less than 0.5% interference in the pre-screen and Z′ values greater than 0.5 in the live pilot screens are observed.

The assays described herein comprise the following advantages. The novel assay disclosed herein eliminates the technical hurdle preventing screening for cGAS modulators and thereby opens up investigation of promising new therapeutic approaches for debilitating and fatal autoimmune diseases and for cancer immunotherapy. Development of cGAMP high-throughput fluorescent and/or bioluminescent assays is novel and could have broad utility for drug discovery and diagnostic applications targeting the cGAS/STING pathway. Additionally, it is possible to add all the coupling enzymes directly to the cGAS reaction and monitor formation of cGAMP in real time.

The invention will be further described in the following examples, which do not limit the scope of the invention described in the claims.

### EXAMPLES

The Examples that follow are illustrative of specific embodiments of the invention, and various uses thereof. They are set forth for explanatory purposes only, and are not to be taken as limiting the invention.

### Example 1

### Development of Assay for Detecting AMP/GMP

AMP/GMP can be detected using an AMP/GMP assay, which can follow the progress of, for example, SVPDE, ENPP1 or any enzyme that produces AMP or GMP (see FIGS. 2-4). Enzyme reaction progress can be indicated by a decrease in the fluorescence polarization. The AMP/GMP detection mixture can comprise an AMP/GMP tracer bound to an AMP/GMP antibody. The tracer can be displaced by AMP or GMP, the invariant product generated during the enzyme reaction. The displaced tracer can freely rotate leading to a decrease in polarization. As a result, the detected AMP/GMP is proportional to a decrease in polarization. The assay can use a far red tracer to minimize interference from fluorescent compounds and light scattering.

### Example 2

### Development of Assay for Detecting Activity of cGAS

cGAS enzyme activity can be detected by using ENPP1 or SVPDE to convert cGAMP to AMP and/or GMP, without also converting ATP and GTP and, simultaneously, converting AMP and GMP to ADP and GDP by using GMP/AMP kinase (see FIG. 2). The detection of ADP can be measured by two assays. In one assay, ADP detection can be carried out by monitoring the consumption of ATP using a luciferase assay (see FIG. 3). In another assay, monitoring of ADP can be carried out by an enzyme-coupled reaction converting ADP to a fluorescent signal (see FIG. 4).

### Example 3

### Development of Assay for Detecting Activity of ADP Based on Consumption of ATP by Luminescence-Assay

Monitoring the consumption of ATP using luciferase is possible, but substrate reduction assays are generally avoided in HTS because of the low signal to background. Thus, an assay can be developed, wherein ATP is formed stoichiometrically with cGAMP (see FIG. 3). Hence, this assay (which is not a substrate reduction assay) is advantageous for HTS assays since there is no concern for low signal to background.

To monitor the increase in ADP by the conversion of ADP to ATP, an ADP converting enzyme, pyruvate kinase and cofactors can be added. The ATP consumption can be detected by adding luciferase enzyme to ATP and luciferin. The luciferase enzyme, which is an ATPase, can convert ATP to AMP and inorganic phosphate (PPi). The luciferin-luciferase reaction can cause an increase in light output. However, over time, decay in light signal can occur, which would correlate with the increased concentrations of ATP used in the experiment. The experiments can be run on plates and placed in a luminometer for detection of light output.

### Example 4

### Development of Homogenous and Non-Radioactive Assay for Detecting Activity of ADP Based on Enzyme-Coupled Reaction Converting ADP to Fluorescent Signal

To monitor the increase in ADP, an enzyme-coupled reaction converting ADP to fluorescent signal can be developed. In this reaction, ADP and phosphopyruvate (PEP) can be converted to hydrogen peroxide (H₂O₂) by pyruvate kinase, cofactors and pyruvate oxidase (see FIG. 4). The hydrogen peroxide can be then combined with a fluorescent dye precursor, reagent Amplex Red (10-acetyl-3,7-dihydroxyphenoxazine) and peroxidase to generate resorufin which emits fluorescence at 590 nm upon excitation at 530 nm. Therefore, with every substrate molecule phosphorylated, an ADP molecule is produced.

### Example 5

### Development and Validation of Biochemical HTS Activity Assays for cGAS

The full-length human cGAS can be synthesized and cloned into a T7 expression vector (GenScript, Piscataway, N.J.) with N-terminal polyhistidine (6xHis) and maltose binding protein (MBP) fusions that can be proteolytically cleaved to produce the native enzyme. The MBP domain increases expression of soluble protein, and the 6xHis tag can be used for affinity purification. The protein can be expressed in a BL21-DE3 strain along with a plasmid that produces human tRNAs that are rare in E. coli using induction with isopropyl β-D-1-thiogalactopyranoside (IPTG). cGAS can be purified using immobilized metal ion affinity chromatography (IMAC) followed by heparin-Sepharose, which binds to the cGAS DNA binding domain. The 6xHis-MBP-domains can be removed by digestion with Tobacco Etch Virus protease, and the native cGAS protein can be separated from the cleavage product on heparin-Sepharose. Purity can be assessed by SDS-PAGE. Enzyme stability can be assessed at 4° C. and through multiple freeze-thaw cycles, and stabilizing agents such as glycerol, DTT, non-ionic detergents or salts can be added if necessary to improve retention of activity.

The assay can be tested with partially purified preparations of cGAS. Initial IMAC chromatography removes small molecules (e.g., nucleotides) that could potentially interfere with the assay, and enzyme eluted from IMAC or heparin-Sepharose can be exchanged into low salt buffer to remove imidazole and other salts from chromatography. Having confirmed detection of exogenously added cGAMP in the cGAS samples, enzyme titrations can be performed; initially using high concentrations of ATP and GTP (e.g., 100-500 μM) and cGAMP formation can be measured using the luminescence-based and fluorescence-based assays. Initial reaction conditions can be as follows: 50 mM KCl, 5 mM Mg(OAc)₂, 50 mM Tris (pH 7.0), 1 mM TCEP, and 0.1 mg/mL 1 BSA, 2 μM dsDNA (22). Subsequently, optimal buffers and additives can be determined, including the size and concentration of dsDNA for supporting maximal cGAS activity.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

### REFERENCES

Klink T. A., et al., Development and validation of a generic fluorescent methyltransferase activity assay based on the transcreener AMP/GMP assay. J Biomol Screen. 2012; 17(1):59-70.

Staeben M., et al., Development and validation of a transcreener assay for detection of AMP- and GMP-producing enzymes. Assay Drug Dev Technol. 2010; 8(3):344-55.

Liu Y., et al., Discovery of acetyl-coenzyme A carboxylase 2 inhibitors: comparison of a fluorescence intensity-based phosphate assay and a fluorescence polarization-based ADP Assay for high-throughput screening. Assay Drug Dev Technol. 2007; 5(2):225-35.

Kleman-Leyer K. M., et al., Characterization and optimization of a red-shifted fluorescence polarization ADP detection assay. Assay Drug Dev Technol. 2009; 7(1):56-67.

Degorce F., et al., HTRF: A technology tailored for drug discovery—a review of theoretical aspects and recent applications. Curr Chem Genomics. 2009; 3:22-32.

Vedvik K. L., et al., Overcoming compound interference in fluorescence polarization-based kinase assays using far-red tracers. Assay Drug Dev Technol. 2004; 2(2):193-203.

Zhang X., et al., The cytosolic DNA sensor cGAS forms an oligomeric complex with DNA and undergoes switch-like conformational changes in the activation loop. Cell Reports. 2014; 6(3):421-30.

Donover P. S., et al., New informatics and automated infrastructure to accelerate new leads discovery by high throughput screening (HTS). Comb Chem High Throughput Screen. 2013; 16(3):180-8.

Cheng N., et al., Development of a Novel Screening Strategy Designed to Discover a New Class of HIV Drugs. J Lab Autom. 2013;19(3):297-303.

Malecka K. A., et al., Identification and characterization of small molecule human papillomavirus E6 inhibitors. ACS Chem Biol. 2014; 9(7):1603-12.

Thompson S., et al., Development of a high-throughput screen for inhibitors of Epstein-Barr virus EBNA1. J Biomol Screen. 2010; 15(9):1107-15.

Fera D., et al., Identification and characterization of small molecule antagonists of pRb inactivation by viral oncoproteins. Chem Biol. 2012; 19(4):518-28.

What is claimed is:

1. A method for measuring G(2′-5′)pA(3′-5′)p (cGAMP) produced in an enzymatically catalyzed reaction, comprising:
(a) contacting, in a first reaction, a biological sample with an enzyme and a first substrate molecule to form a first product;
(b) contacting, in a second reaction, the first product with a second substrate molecule in the presence of a first catalytically active enzyme to form a second product and a third product; and
(c) detecting the second and the third products by an antibody;
thereby measuring the produced cGAMP.

2. The method of claim 1, wherein:
(a) the first substrate molecule is adenosine triphosphate (ATP) and/or guanosine triphosphate (GTP);
(b) the second substrate molecule comprises a phosphate molecule;
(c) the first catalytically active enzyme is a phosphodiesterase, comprising snake venom phosphodiesterase-1 (SVPDE) and/or ectonucleotide pyrophosphatase/phosphodiesterase 1 (ENPP1);
(d) the enzyme catalyzing step (a) comprises cyclic GMP-AMP synthase (cGAS) activity;
(e) the first product is cyclic guanosine monophosphate-adenosine monophosphate (cGAMP);
(f) the second product is adenosine monophosphate (AMP);
(g) the third product is guanosine monophosphate (GMP); and
(h) the antibody is an AMP/GMP-specific antibody.

3. The method of claim 1, further comprising:
(d) contacting, in a third reaction, the second product and the third product in the presence of a second catalytically active enzyme to form a fourth product and a fifth product.

4. The method of claim 3, wherein:
(a) the second catalytically active enzyme comprises AMP/GMP kinase;
(b) the fourth product is adenosine diphosphate (ADP); and
(c) the fifth product is guanosine diphosphate (GDP).

5. The method of claim 3, wherein the formation of the fourth product is detected by:
(a) contacting, in a first reaction, the fourth product and a third substrate molecule in the presence of a third catalytically active enzyme to form a sixth product;
(b) contacting, in a second reaction, the sixth product with a bioluminescent agent and/or a fluorescent agent in the presence of a fourth catalytically active enzyme;
thereby generating a signal, comprising a bioluminescent signal and/or a fluorescent signal; and
(c) measuring the signal.

6. The method of claim 5, wherein:
(a) the third substrate molecule is phosphoenolpyruvate;
(b) the third catalytically active enzyme is pyruvate kinase and/or pyruvate oxidase;
(c) the sixth product is adenosine triphosphate (ATP) and/or hydrogen peroxide;
(d) the fourth catalytically active enzyme is luciferase;
(e) the bioluminescent agent is luciferin; and
(f) the fluorescent agent comprises Amplex Red, fluorescein, mCherry fluorescent protein, cyanine dyes, TRITC, pacific blue, and/or pacific orange.

7. The method of claim 3, further comprising detecting cGAMP in the biological sample;
thereby measuring cGAMP produced in the reaction.

8. The method of claim 1, wherein the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

9. A method for measuring cyclic GMP-AMP synthase (cGAS) activity, comprising:
(a) contacting, in a first reaction, a biological sample with an enzyme and a first substrate molecule to form a first product; and
(b) contacting, in a second reaction, the first product with a second substrate molecule in the presence of a first catalytically active enzyme to form a second product and a third product; and
(c) detecting the second and the third products by an antibody;
thereby measuring the cGAS activity.

10. The method of claim 9, wherein:
(a) the first substrate molecule is adenosine triphosphate (ATP) and/or guanosine triphosphate (GTP);
(b) the second substrate molecule comprises a phosphate molecule;
(c) the first catalytically active enzyme is a phosphodiesterase, comprising snake venom phosphodiesterase-1 (SVPDE) and/or ectonucleotide pyrophosphatase/phosphodiesterase 1 (ENPP1);
(d) the enzyme catalyzing step (a) comprises cyclic GMP-AMP synthase (cGAS) activity;
(e) the first product is cyclic guanosine monophosphate-adenosine monophosphate (cGAMP);
(f) the second product is adenosine monophosphate (AMP);
(g) the third product is guanosine monophosphate (GMP); and
(h) the antibody is an AMP/GMP-specific antibody.

11. The method of claim 9, further comprising:
(d) contacting, in a third reaction, the second product and the third product in the presence of a second catalytically active enzyme to form a fourth product and a fifth product.

12. The method of claim 11, wherein:
(a) the second catalytically active enzyme comprises AMP/GMP kinase;
(b) the fourth product is adenosine diphosphate (ADP); and
(c) the fifth product is guanosine diphosphate (GDP).

13. The method of claim 11, wherein the formation of the fourth product is detected by:
(a) contacting, in a first reaction, the fourth product and a third substrate molecule in the presence of a third catalytically active enzyme to form a sixth product;
(b) contacting, in a second reaction, the sixth product with a bioluminescent agent and/or a fluorescent agent in the presence of a fourth catalytically active enzyme;
thereby generating a signal, comprising a bioluminescent signal and/or a fluorescent signal; and
(c) measuring the signal.

14. The method of claim 13, wherein:
(a) the third substrate molecule is phosphoenolpyruvate;
(b) the third catalytically active enzyme is pyruvate kinase and/or pyruvate oxidase;
(c) the sixth product is adenosine triphosphate (ATP) and/or hydrogen peroxide;
(d) the fourth catalytically active enzyme is luciferase;
(e) the bioluminescent agent is luciferin; and
(f) the fluorescent agent comprises Amplex Red, fluorescein, mCherry fluorescent protein, cyanine dyes, tetramethylrhodamine (TRITC), pacific blue, and/or pacific orange.

15. The method of claim 11, further comprising detecting cGAMP in the biological sample; thereby measuring cyclic GMP-AMP synthase (cGAS) activity.

16. The method of claim 9, wherein the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

17. A method for measuring G(2′-5′)pA(3′-5′)p (cGAMP) produced in an enzymatically catalyzed reaction and/or cyclic GMP-AMP synthase (cGAS) activity, comprising:
(a) contacting a biological sample with a substrate molecule and an agent in the presence of a catalytically active enzyme;
(b) measuring a signal; and
(c) detecting cGAMP in the biological sample; thereby measuring cGAMP produced in the reaction and/or cyclic GMP-AMP synthase (cGAS) activity.

18. The method of claim 17, wherein:
(a) the substrate molecule comprises adenosine triphosphate (ATP), guanosine triphosphate (GTP), a phosphate molecule, and/or phosphoenolpyruvate;
(b) the catalytically active enzyme comprises a phosphodiesterase, AMP/GMP kinase, pyruvate kinase, pyruvate oxidase, luciferase, and/or peroxidase;
(c) the agent comprises a bioluminescent agent, comprising luciferin or a fluorescent agent, comprising Amplex Red; and
(d) the signal is a bioluminescent signal or a fluorescent signal.

19. The method of claim 17, wherein the method is:
(a) a competitive assay method;
(b) a homogenous assay method;
(c) an assay method having low nanomolar sensitivity;
(d) an assay method allowing for direct detection of cGAMP;
(e) an assay method operable in endpoint or continuous mode; and/or
(f) a high-throughput screening (HTS) assay method.

20. A kit for:
(a) detecting and measuring cGAMP produced in an enzymatically catalyzed reaction, comprising adenosine triphosphate (ATP), guanosine triphosphate (GTP), a phosphate molecule, phosphoenolpyruvate, a phosphodiesterase, AMP/GMP kinase, pyruvate kinase, luciferase, and a bioluminescent agent;
(b) detecting and measuring cGAMP produced in an enzymatically catalyzed reaction, comprising adenosine triphosphate (ATP), guanosine triphosphate (GTP), a phosphate molecule, phosphoenolpyruvate, a phosphodiesterase, AMP/GMP kinase, pyruvate kinase, pyruvate oxidase, peroxidase, and a fluorescent agent;
(c) measuring cyclic GMP-AMP synthase (cGAS) activity, comprising adenosine triphosphate (ATP), guanosine triphosphate (GTP), a phosphate molecule, phosphoenolpyruvate, a phosphodiesterase, AMP/GMP kinase, pyruvate kinase, luciferase, and a bioluminescent agent;
(d) measuring cyclic GMP-AMP synthase (cGAS) activity, comprising adenosine triphosphate (ATP), guanosine triphosphate (GTP), a phosphate molecule, phosphoenolpyruvate, a phosphodiesterase, AMP/GMP kinase, pyruvate kinase, pyruvate oxidase, peroxidase, and a fluorescent agent;
(e) detecting and measuring cGAMP produced in an enzymatically catalyzed reaction, comprising an antibody against AMP/GMP, wherein the antibody has the ability to preferentially recognize AMP/GMP in the presence of substrate molecules, and wherein the antibody specifically binds AMP/GMP; and/or
(f) measuring cyclic GMP-AMP synthase (cGAS) activity, comprising an antibody against AMP/GMP, wherein the antibody has the ability to preferentially recognize AMP/GMP in the presence of substrate molecules, and wherein the antibody specifically binds AMP/GMP.
